(19) United States
(12) Patent Application Publication
(10) Pub. No.: US 2018/0344927 A1
(43) Pub. Date: Dec. 6, 2018

(54) DRUG DELIVERY APPARATUS AND SYSTEM, METHODS OF FORMING AND OPERATING THE SAME

(71) Applicant: SINGAPORE HEALTH SERVICES PTE LTD, Singapore (SG)

(72) Inventor: Shariq Ali Khan, Singapore (SG)

(21) Appl. No.: 15/776,776

(22) PCT Filed: Nov. 16, 2016
(86) PCT No.: PCT/SG2016/050566

§ 371 Date: May 16, 2018

(30) Foreign Application Priority Data
Nov. 18, 2015 (SG) 10201509495Y

(51) Int. Cl. A61M 5/168; A61M 5/20; A61M 5/14; A61M 39/28; G16H 20/17; G16H 10/60

(52) U.S. Cl. CPC A61M 5/16813; A61M 2205/50; A61M 5/1413; A61M 39/281; G16H 20/17; G16H 10/60; A61M 2207/00; A61M 2205/3306; A61M 2205/14; A61M 2205/3584; A61M 2205/6072; A61M 2205/3553; A61M 2202/048; A61M 2205/6009; A61M 5/20

(57) ABSTRACT

Various embodiments may provide a drug delivery apparatus for coupling with a container. The drug delivery apparatus may include a fluid conduit, a sensor configured to determine the coupling of the container to the drug delivery apparatus, and a flow controller configured to switch between an open state which allows fluid flow through the fluid conduit to a subject, and a closed state which prevents fluid flow through the fluid conduit to the subject. The drug delivery apparatus may additionally include a communication module configured to communicate via wireless means with a remote drug identification apparatus, and a processing module electrically coupled to the communication module and to the flow controller.

### CROSS-REFERENCE TO RELATED APPLICATION

This application claims the benefit of priority of Singapore application No. 10201509495Y filed Nov. 18, 2015, the contents of it being hereby incorporated by reference in its entirety for all purposes.

### TECHNICAL FIELD

Various aspects of this disclosure relate to drug delivery apparatuses and/or systems. Various aspects of this disclosure relate to methods for forming and/or operating the same.

### BACKGROUND

Currently, medications used in the operation theatre (OT) are manually filled into syringes, hand labelled and delivered by an anesthesiologist without any secondary check. The anesthesia medication error rate is about 0.75%. With more than 200 million anesthetics delivered annually, the number of anesthesia medication errors may be more than a million each year. The annual costs of medication errors due to anesthesia may amount to more than USD 6 billion in the United States. It has also been reported that more than 770,000 injuries and deaths are caused by adverse drug events. Adverse drug events cost each hospital USD 5.6 million of which USD 2.8 million may be preventable. In Canada, the annual cost of medication-related errors is about CAD 17 billion to about CAD 29 billion annually.

About 60% or more of anesthesia medication errors are substitution errors. In an anonymous survey of 97 anesthesiologists, it has been found that more than 66% of the anesthesiologists have committed a medication error in the operating theatre (OT), and about 45% of the errors were ampoule swaps and syringe swaps. FIG. 1A is a schematic 100a illustrating ampoule swaps and syringe swaps. An ampoule swap occurs when medication from an incorrect ampoule is loaded into a syringe, while a syringe swap occurs when medication from an incorrect ampoule is delivered to a patient. FIG. 1B is a table 100b showing the numbers and percentage (%) the causes of drug preparation or syringe errors out of a total of 452 reported to the Australian Anesthetic Incident Monitoring Study. The 452 cases constitute about 50% of all drug errors. FIG. 1C is a table 100c showing the major factors contributing to syringe or drug preparation errors reported to the Australian Anesthetic Incident Monitoring Study. An incident may have multiple contributing factors. The values are the actual numbers while the figures in percentage represent the proportion.

Existing devices (e.g. Codonics label-printer) help to reduce medication errors in the OT by reading barcodes on drug ampoules to identify the drug, and to print correct labels for the drug to be adhered to the syringe, thereby reducing ampoule swaps. However, studies suggest that these devices do not facilitate scanning of a drug-filled syringe prior to administration. Accordingly, syringe swaps remain largely unresolved by such existing devices.

### SUMMARY

Various embodiments may provide a drug delivery apparatus for coupling with a container, such as a syringe, according to various embodiments. The drug delivery apparatus may include a fluid conduit. The drug delivery apparatus may further include a sensor configured to determine the coupling of the container to the drug delivery apparatus. The drug delivery apparatus may also include a flow controller configured to switch between an open state which allows fluid flow through the fluid conduit to a subject, and a closed state which prevents fluid flow through the fluid conduit to the subject. The drug delivery apparatus may additionally include a communication module configured to communicate via wireless means with a remote drug identification apparatus. The drug delivery apparatus may further include a processing module electrically coupled to the communication module and to the flow controller. The flow controller may be configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus. The flow controller may be configured to be switched to the closed state after receiving a trigger from the sensor upon the container being decoupled from the drug delivery apparatus.

Various embodiments may provide a method of operating a drug delivery apparatus may be provided according to various embodiments. The method may include coupling the drug delivery apparatus with a container. The drug delivery apparatus may include a fluid conduit. The drug delivery apparatus may further include a sensor configured to determine the coupling of the container to the drug delivery apparatus. The drug delivery apparatus may also include a flow controller configured to switch between an open state which allows fluid flow through the fluid conduit to a subject, and a closed state which prevents fluid flow through the fluid conduit to the subject. The drug delivery apparatus may additionally include a communication module configured to communicate via wireless means with a remote drug identification apparatus. The drug delivery apparatus may also include a processing module electrically coupled to the communication module and to the flow controller. The flow controller may be configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus. The method may further include decoupling the container and the drug delivery apparatus, wherein the flow controller is configured to be switched to the closed state after receiving a trigger from the sensor upon the container being decoupled from the drug delivery apparatus.

Various embodiments may provide a method of forming a drug delivery apparatus according to various embodiments. The method may include providing a fluid conduit. The method may also include providing a sensor configured to determine the coupling of the container to the drug delivery apparatus. The method may further include providing a flow controller configured to switch between an open state which allows fluid flow through the fluid conduit to a subject, and a closed state which prevents fluid flow through the fluid conduit to the subject. The method may additionally include providing a communication module configured to communicate via wireless means with a remote drug identification apparatus. The method may further include electrically coupling a processing module to the communication module and to the flow controller. The flow controller may be configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus. The flow controller may be configured to be switched to the closed state after receiving a trigger from the sensor upon the container being decoupled from the drug delivery apparatus.

### BRIEF DESCRIPTION OF THE DRAWINGS

The invention will be better understood with reference to the detailed description when considered in conjunction with the non-limiting examples and the accompanying drawings, in which:

FIG. 1A is a schematic illustrating ampoule swaps and syringe swaps.

FIG. 1B is a table showing the numbers and percentage (%) the causes of drug preparation or syringe errors out of a total of 452 reported to the Australian Anesthetic Incident Monitoring Study.

FIG. 1C is a table showing the major factors contributing to syringe or drug preparation errors reported to the Australian Anesthetic Incident Monitoring Study.

FIG. 2 is a schematic of a drug delivery apparatus for coupling with a container, such as a syringe, according to various embodiments.

FIG. 3 shows a schematic illustrating a method of operating a drug delivery apparatus according to various embodiments.

FIG. 4 is a schematic showing a method of operating a drug delivery system according to various embodiments.

FIG. 5 is a schematic illustrating a method of forming a drug delivery apparatus according to various embodiments.

FIG. 6A shows a fluid conduit and a flow control valve of a drug delivery apparatus according to various embodiments.

FIG. 6B shows various components included in a housing unit of the drug delivery apparatus according to various embodiments.

FIG. 6C shows the assembled housing unit of the drug delivery apparatus according to various embodiments.

FIG. 6D shows the technical drawings of the housing unit according to various embodiments.

FIG. 6E shows a photo of a Bluetooth module included in the drug delivery apparatus according to various embodiments.

FIG. 6F shows a schematic of the Bluetooth module included in the drug delivery apparatus according to various embodiments.

FIG. 6G shows images of the side view (left) and the perspective view (right) of the servo motor of the drug delivery apparatus according to various embodiments.

FIG. 6H shows the Arduino microcontroller included in the drug delivery apparatus according to various embodiments.

FIG. 6I shows the battery and charging unit included in the drug delivery apparatus according to various embodiments.

FIG. 6J shows a drug identification apparatus configured to work with the drug delivery apparatus according to various embodiments.

FIG. 7 shows a pinch valve of a drug delivery apparatus according to various embodiments.

FIG. 8A shows the drug delivery apparatus according to various embodiments with the flow controller in a closed state.

FIG. 8B shows a barcode scanner being used to scan a barcode on a syringe according to various embodiments.

FIG. 8C shows a computing device with a software being used to identify the drug based on the barcode and to generate an audio-visual indication containing the name of the drug as well as the concentration of the drug according to various embodiments.

FIG. 8D shows the drug delivery apparatus according to various embodiments in a drug acceptance state after the computing device with the software transmitting a signal containing a positive indication to the drug delivery apparatus.

FIG. 8E shows the syringe being brought into proximity of the drug delivery apparatus according to various embodiments.

FIG. 9A is a housing unit of a drug delivery apparatus according to various embodiments.

FIG. 9B shows a customized disposable tubing of the drug delivery apparatus according to various embodiments.

FIG. 9C shows the integration of the housing unit and the customized disposable tubing according to various embodiments.

FIG. 9D shows a cross-sectional schematic of the housing unit and the customized disposable tubing in which the pins are engaged with the slider cover of the drug delivery apparatus according to various embodiments.

FIG. 9E shows a cross-sectional schematic of the housing unit and the customized disposable tubing in which the pins are disengaged from the slider cover of the drug delivery apparatus according to various embodiments.

FIG. 9F is a schematic of a side view of the slider cover and the customized disposable tubing in which the cover is at a first position so that the opening of the cover is in fluidic communication with the port on the tubing according to various embodiments.

FIG. 9G is a schematic of a side view of the slider cover and the customized disposable tubing in which the cover is at a second position so that the opening of the cover is not in fluidic communication with the port on the tubing according to various embodiments.

FIG. 9H shows a method of operating a drug delivery system including a drug delivery apparatus and a drug identification apparatus according to various embodiments.

FIG. 10 shows images of a prototype system according to various embodiments used in conjunction with a Codonics SLS Label printer.

FIG. 11 is a table showing the results of the study.

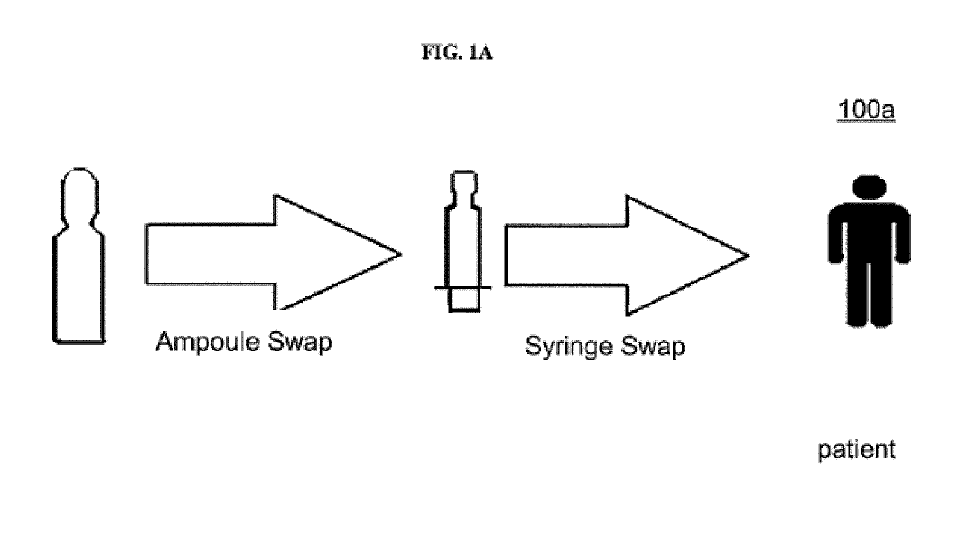

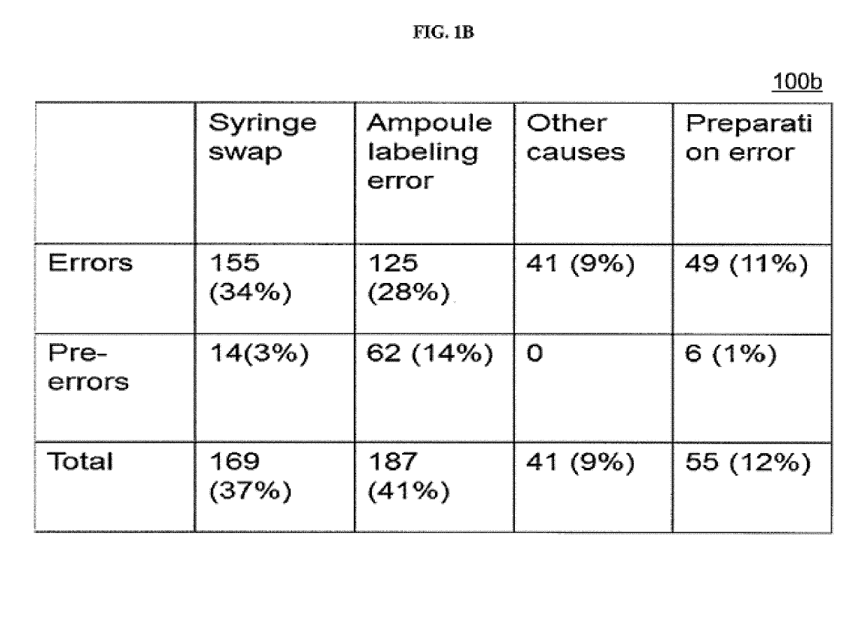

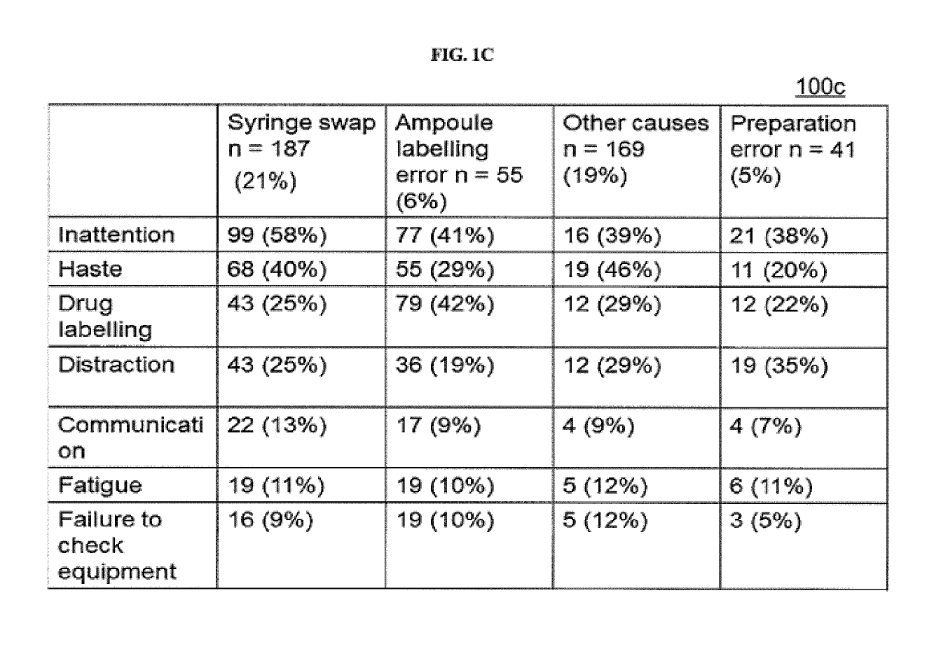

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

Embodiments described in the context of one of the methods or apparatuses/systems are analogously valid for the other methods or apparatuses/systems. Similarly, embodiments described in the context of a method are analogously valid for an apparatus or a system, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The system or apparatus as described herein may be operable in various orientations, and thus it should be understood that the terms “top”, “bottom”, etc., when used in the following description are used for convenience and to aid understanding of relative positions or directions, and not intended to limit the orientation of the system or apparatus.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

Various embodiments may seek to resolve or alleviate the abovementioned problems. Various embodiments may seek to prevent or reduce syringe swaps.

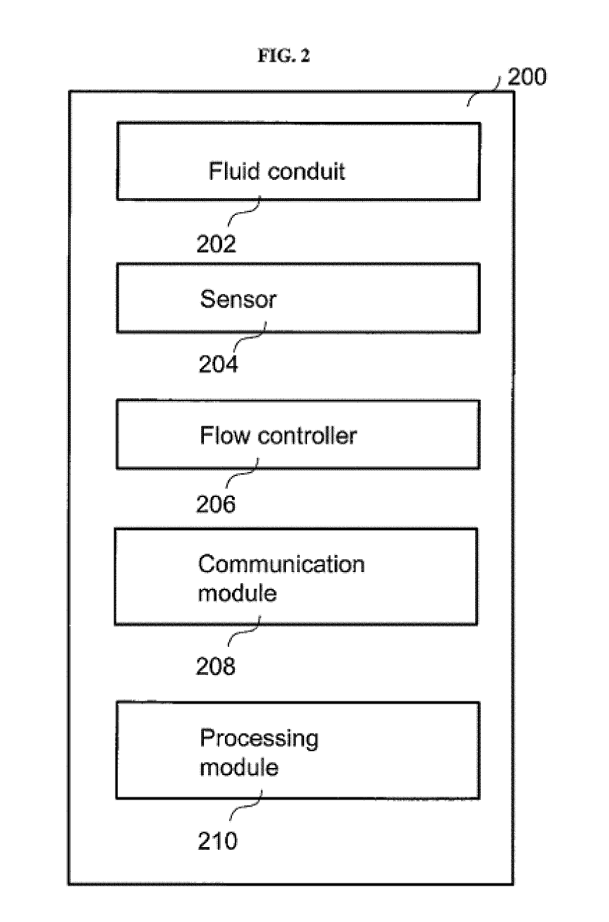

FIG. 2 is a schematic of a drug delivery apparatus 200 for coupling with a container, such as a syringe, according to various embodiments. The drug delivery apparatus 200 may include a fluid conduit 202. The drug delivery apparatus 200 may further include a sensor 204 configured to determine the coupling of the container to the drug delivery apparatus 200. The drug delivery apparatus 200 may also include a flow controller 206 configured to switch between an open state which allows fluid flow through the fluid conduit 202 to a subject, and a closed state which prevents fluid flow through the fluid conduit 202 to the subject. The drug delivery apparatus 200 may additionally include a communication module 208 configured to communicate via wireless means with a remote drug identification apparatus. The drug delivery apparatus 200 may further include a processing module 210 electrically coupled to the communication module 208 and to the flow controller 206. The flow controller 206 may be configured to be switched to the open state after receiving a control signal from the processing module 210 upon the communication module 208 receiving a positive indication from the drug identification apparatus that the container comprises a drug that is to be delivered to the subject, so that the drug is allowed to flow from the container through the fluid conduit 202 to the subject when the container is coupled to the drug delivery apparatus 200. The flow controller 206 may be configured to be switched to the closed state after receiving a trigger from the sensor 204 upon the container being decoupled from the drug delivery apparatus 200.

In other words, the drug delivery apparatus 200 may include a fluid conduit for injecting a drug to a subject. The drug delivery apparatus 200 may include a flow controller 206 which acts as a switch to allow or stop the flow of the drug to the subject. The flow controller 206 may be configured to be switched to an open state only when the drug delivery apparatus 200 receives a positive indication from a drug identification apparatus indicating that the drug that in a container is the correct drug intended to be administered to the subject. The drug identification apparatus may transmit the positive indication to the communication module 208, and the communication module 208 upon receiving the positive indication may inform the processing module 210, which in turn may transmit a control signal to the flow controller 206 to switch or allow the switching of the flow controller 206 from the closed state to the open state. The container may then be coupled to the drug delivery apparatus 200. After administration of the drug, a sensor 204 may sense the decoupling of the container from the drug delivery apparatus 200. The sensor 204 may then trigger the flow controller to switch or allow the switching of the flow controller 206 to the closed state. The flow controller 206 may then be switched from the open state back to the closed state.

Various embodiments may thus act as a safety lock which allows drug administration to the subject, e.g. a patient, only when it has been confirmed that the drug in a container, such as a syringe, is indeed the correct drug that is intended to be delivered to the subject. Various embodiments may seek to address the abovementioned issues. In various embodiments, the various components such as the fluid conduit 202, the sensor 204, the flow controller 206, the communication module 208, and the processing module 210 may cooperate with one another to provide a drug delivery apparatus 200 that is convenient for an user, such as an anesthesiologist or a doctor, to administer drugs to a subject, such as a patient. In various embodiments, the drug delivery apparatus 200 may cooperate with a drug identification system to provide an effective system for identifying and ensuring that the correct drugs are administered.

In various embodiments, the fluid conduit 202 may be or may include an intravenous tubing, such as a disposable plastic intravenous tubing. The sensor 204 may be referred to as an interrupter sensor.

In the current context, the drug may refer to a medication or substance to be delivered to the subject via injection or intravenous means.

In various embodiments, coupling the container with the drug delivery apparatus 200 may include attaching the container to the drug delivery apparatus 200 or moving the container to be in contact or near the drug delivery apparatus 200 so that the drug comprised in the container flows at least partially into the drug delivery apparatus 200. Decoupling the container from the drug delivery apparatus 200 may include detaching or removing the container from the drug delivery apparatus 200.

In various embodiments, the flow controller 206 may include a cover having an opening. The cover may be at a first position in which the opening of the cover is in fluidic communication with a port, i.e. an intravenous drug administration port, on the fluid conduit when the flow controller is in the open state. The cover may be at a second position in which the opening of the cover is not in fluidic communication with the port on the fluid conduit when the flow controller is in the closed state. In other words, the opening of the cover may be aligned with the port on the fluid conduit 202 when the flow controller 206 is in the open state, and may be misaligned with the port on the fluid conduit when the flow controller 206 is in the closed state. The flow controller 206 may also be referred to as a flow control mechanism.

The flow controller 206 may include a lock. The lock may also be referred to as a locking mechanism. The locking mechanism may include an actuator, such as a motor, electrically coupled to the processing module 210. The locking mechanism may further include a pair of pins or jaws coupled to the actuator. The pair of pins or jaws may be engaged with the cover when the flow controller 206 is in the closed state, thus ‘locking’ or securing the cover in the second position and preventing the flow of fluid through the fluid conduit to the subject.

The processing module 210 may be configured to control the lock or locking mechanism, e.g. the pins or jaws, to disengage from the cover upon the communication module 208 receiving the positive indication from the drug identification apparatus so that the cover is allowed or configured to be switched manually to the open state. When the processing module 210 receives the positive indication from the drug identification apparatus via the communication module 208, the processing module 210 may transmit the control signal to control the actuator to move the pair of pins or jaws to disengage from the cover. The user such as an anesthesiologist or a doctor, may then move the cover manually to the first position in which the cover is in fluidic communication with the port on the fluid conduit 202. The flow controller 206 may thus be configured to be switched manually to the open state after receiving the control signal from the processing module 210, wherein the control signal is transmitted from the processing module 210 to the flow controller 206 upon the communication module receiving a positive indication from the drug identification apparatus.

The sensor 204 may be a mechanical sensor. The mechanical sensor may include a spring configured to control the flow controller 206 to switch to the closed state after the container is decoupled from the drug delivery apparatus 200. When the user, e.g. the anesthesiologist or the doctor, moves the cover from the second position to the first position, the spring may be actuated from an uncompressed state to a compressed state. The spring may be prevented from returning to the uncompressed state by the container when the container is coupled to the drug delivery apparatus 200. When the container is decoupled from the drug delivery apparatus 200, the spring may sense the decoupling and may return to the uncompressed state, thus mechanically controlling or triggering the cover to move from the first position back to the second position. The flow controller 206 may thus be configured to be switched automatically and directly to the closed state after receiving the trigger from the sensor 204.

In various embodiments, the processing module 210 may be configured to control the flow controller 206, e.g. the lock of the flow controller 206, to unlock for enabling the switch from the closed state to the open state via the control signal, so that the flow controller 206 may latter be configured or allowed to be switched manually to the open state. In various other embodiments, the flow controller 206 may be configured to be switched from the closed state to the open state directly and automatically by the processing module 210 via the control signal. The flow controller 206 may be configured to be switched from the closed state to the open state automatically, without manual inputs or intervention from the user. The control signal may be transmitted from the processing module 210 to the flow controller 206.

In various embodiments, the sensor 204 may be configured to trigger the flow controller 206 so that the flow controller 206 may be configured or allowed to be switched manually to the closed state. In various other embodiments, the flow controller 206 may be configured to be switched directly by the sensor 204 via the trigger. In various embodiments, the trigger may be a mechanical trigger, while in various other embodiments, the trigger may be a further control signal. In various other embodiments, the sensor 204 may be configured to trigger the processing module 210, which in turns trigger the flow controller so that that the flow controller 206 may be allowed to be switched manually or may be configured to switch automatically to the closed state.

In various embodiments, the flow controller 206 may be configured to be switched to the open state after receiving the control signal and after receiving a trigger from the sensor that the container, e.g. a syringe is within a predetermined distance from the drug delivery apparatus 200. The predetermined distance may for instance, be any value in the range of about 1 cm to about 10 cm from the drug delivery apparatus 200. The predetermined distance may be about 5 cm from the drug delivery apparatus 200.

In various embodiments, the flow controller 206 may include a flow control valve configured to switch between the open state and the closed state. The flow controller 206 may further include an electromagnet configured to control the flow control valve. The processing module 210 may be configured to control the electromagnet via the control signal to open the flow control valve so that the flow control valve switches to the open state upon the communication module 208 receiving the positive indication from the drug identification apparatus. The flow control valve may be configured to be switched to the open state by the electromagnet after the electromagnet receiving the control signal from the processing module 210.

In various embodiments, the flow controller 206 may include a pinch valve configured to switch between the open state and the closed state. The pinch valve may be configured to be switched to the open state after receiving the control signal from the processing module 210 upon the communication module 208 receiving the positive indication from the drug identification apparatus 200. In other words, the communication module 208 may send a signal to the processing module 210 upon the communication module 208 receiving the positive indication from the drug identification apparatus 200, and when the processing module 210 receives the signal, the processing module 210 may send the control signal to the pinch valve to control the pinch valve to switch from the closed state to an open state. The flow controller 206 may further include an electromagnet to control the pinch valve. The flow controller 206 may further include a biasing spring to cooperate with the electromagnet to control the pinch valve.

The sensor 204 may be an optical sensor such as an infrared sensor. The processing module 208 may be electrically coupled to the optical sensor. The sensor 204 may be configured to determine the coupling of the container to the drug delivery apparatus 200, i.e. whether the container is coupled to the drug delivery apparatus 200.

The sensor 204 may be configured to control or trigger the flow controller, e.g. the flow control valve or the pinch valve to switch to the closed state after the container is decoupled from the drug delivery apparatus.

The flow controller 206 may be configured to remain in the closed state by the processing module 210 upon the communication module 208 receiving a negative indication from the drug identification apparatus that the container includes another drug that is not intended to be delivered to the subject, i.e. the container contains the wrong drug, or when the communication module 208 does not receive any indication from the drug identification apparatus. The processing module 208 may be configured to not transmit the control signal to the flow controller 206 so that the flow controller 206 is disallowed or prevented to be switched to an open state upon the communication module 208 receiving a negative indication from the drug identification apparatus, or when the communication module 208 does not receive any indication from the drug identification apparatus.

In various embodiments, the communication module 208 may be configured to transmit a signal to the remote drug identification apparatus indicating completion of drug delivery after the container is decoupled from the drug delivery apparatus. When the sensor 204 determines that the container has been decoupled from the drug delivery apparatus 200, the sensor 204 may inform the processing module 208. The processing module 208 may then control the communication module 208 to transmit the signal to the remote drug identification apparatus indicating completion of drug delivery.

In various embodiments, the communication module 208 may be or may include a transceiver. The communication module 208 may include a receiver and/or a transmitter. Using wireless communications for communication between the drug delivery apparatus 200 and the remote drug identification apparatus may improve the convenience of using the drug delivery apparatus 200.

The wireless means may be, but is not limited to, any one selected from a group consisting of Bluetooth, Wi-Fi, and Zigbee. Other examples of wireless communications may include fourth generation of wireless mobile telecommunications technology (4G), third generation of wireless mobile telecommunications technology (3G), or Light Fidelity (Li-Fi) technology. Using wireless means to communicate with the remote drug identification apparatus may allow the drug delivery apparatus 200 to communicate more effectively and conveniently with the remote drug identification system.

The processing module 210 may be or may include a processor or a processing circuit. A “circuit” may be understood as any kind of a logic implementing entity, which may be special purpose circuitry or a processor executing software stored in a memory, firmware, or any combination thereof. Thus, in various embodiments, a “circuit” may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). A “circuit” may also be a processor executing software, e.g. any kind of computer program, e.g. a computer program using a virtual machine code such as e.g. Java. Any other kind of implementation of the respective functions which will be described in more detail below may also be understood as a “circuit” in accordance with an alternative embodiment.

In various embodiments, a drug delivery system including a drug delivery apparatus 200 and a remote drug identification apparatus may be provided. In various embodiments, the drug delivery apparatus 200 and the remote identification apparatus may cooperate with each other to effectively identify drugs and prevent syringe swaps.

The remote drug identification apparatus may include a scanner configured to scan a label, e.g. a barcode, on the container to determine, based on the label, an identity of a drug comprised in the container.

The remote drug identification apparatus may further include a computing device configured to determine whether the identified drug comprised in the container matches with the drug that is to be delivered to the subject. The computer may be coupled by wired or wireless means to the scanner. The computer device may be a personal computer, a tablet computer, or any processing device including a processor or processing circuit.

The remote drug identification apparatus may receive an input or information on the drug that is to be delivered to the subject. For instance, the remote drug identification apparatus may receive a record from a database such as Anesthesia Electronic Records database. The remote drug identification apparatus may be configured to transmit the positive indication to the drug delivery apparatus when the identified drug comprised in the container matches with the drug that is to be delivered to the subject.

In various embodiments, determining whether the container comprises a drug that is to be delivered to the subject also includes determining whether the drug is suitable for the subject based on a history of drug allergies of the subject. The history may be stored in the database and may be retrieved by the remote drug identification apparatus before administration of the drug to the subject.

The remote drug identification apparatus may be configured to provide an output providing details of the identified drug. The output may be an audio output, a visual output or an output including audio and visual components, i.e. an audio-visual output. The output may be provided after scanning the label, and before coupling of the container to the drug delivery apparatus 200. The output may provide an user, such as the an anesthesiologist or a doctor administering the drug, with the identity of the drug in the container, and may help the user determine whether the drug in the container is indeed the drug to be administered. The drug delivery apparatus 200 may further ensure that the drug administered to the subject is the correct drug that is intended to be administered. The details of the drug may include one or more selected from a group consisting of the name of the drug, the dosage of the drug, whether the identified drug matches with the information of the drug on the record or database, and whether the subject is allergic to the identified drug.

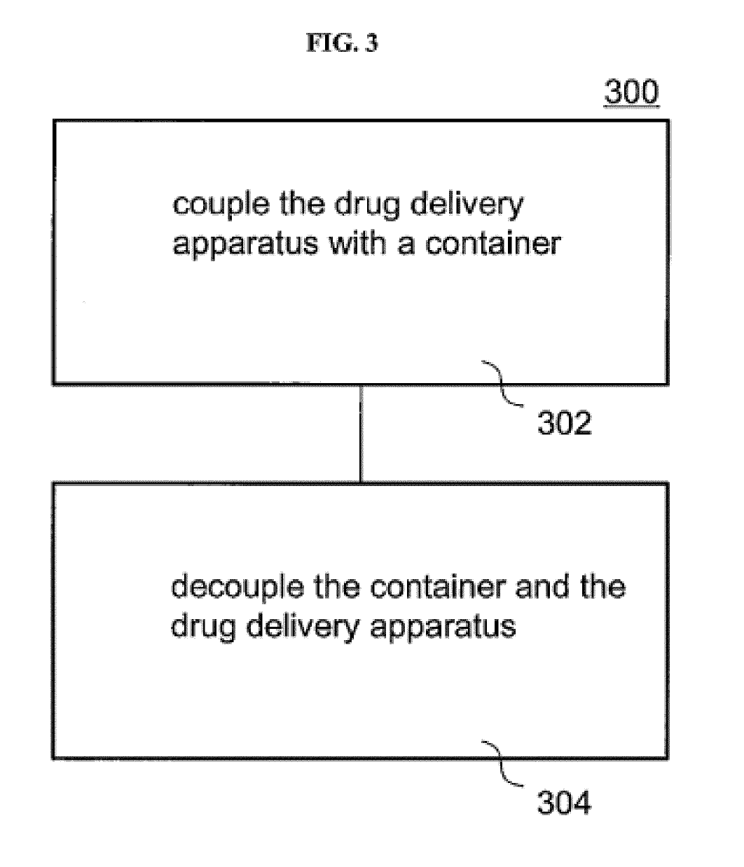

In various embodiments, a method of operating a drug delivery apparatus may be provided according to various embodiments. FIG. 3 shows a schematic 300 illustrating a method of operating a drug delivery apparatus according to various embodiments. The method may include, in 302, coupling the drug delivery apparatus with a container. The drug delivery apparatus may include a fluid conduit. The drug delivery apparatus may further include a sensor configured to determine the coupling of the container to the drug delivery apparatus. The drug delivery apparatus may also include a flow controller configured to switch between an open state which allows fluid flow through the fluid conduit to a subject, and a closed state which prevents fluid flow through the fluid conduit to the subject. The drug delivery apparatus may additionally include a communication module configured to communicate via wireless means with a remote drug identification apparatus. The drug delivery apparatus may also include a processing module electrically coupled to the communication module and to the flow controller. The flow controller may be configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus. The method may further include, in 304, decoupling the container and the drug delivery apparatus, wherein the flow controller is configured to be switched to the closed state after receiving a trigger from the sensor upon the container being decoupled from the drug delivery apparatus.

In other words, a container comprising the drug may be coupled to the drug delivery apparatus as described herein. The drug delivery apparatus may be configured to allow the drug in the container to flow through the fluid conduit to the subject after the drug delivery apparatus receives an indication from a remote drug identification apparatus that the drug is indeed intended for the subject. Upon the drug delivery apparatus receiving the indication from the remote drug identification apparatus that the drug is indeed intended for the subject, the flow controller of the drug delivery apparatus may be switched automatically or manually to the open state, thus allowing the drug to flow from the container through the fluid conduit to the subject. After delivery of the drug, the sensor may detect that the container is being decoupled from the drug delivery apparatus, and may provide a trigger to the flow controller so that the flow controller may be switched back to the closed state automatically or manually.

When the flow controller is switched manually, the control signal provided by the processing module or the trigger provided by the sensor may unlock the flow controller, and/or may enable or allow the manual switching to be carried out after the unlocking.

The method may further include coupling the drug delivery apparatus to the subject. The fluid conduit may have an end that is configured to be inserted onto a blood vessel, e.g. a vein of the subject. The drug delivery apparatus may be coupled to the subject before coupling the container with the drug delivery apparatus.

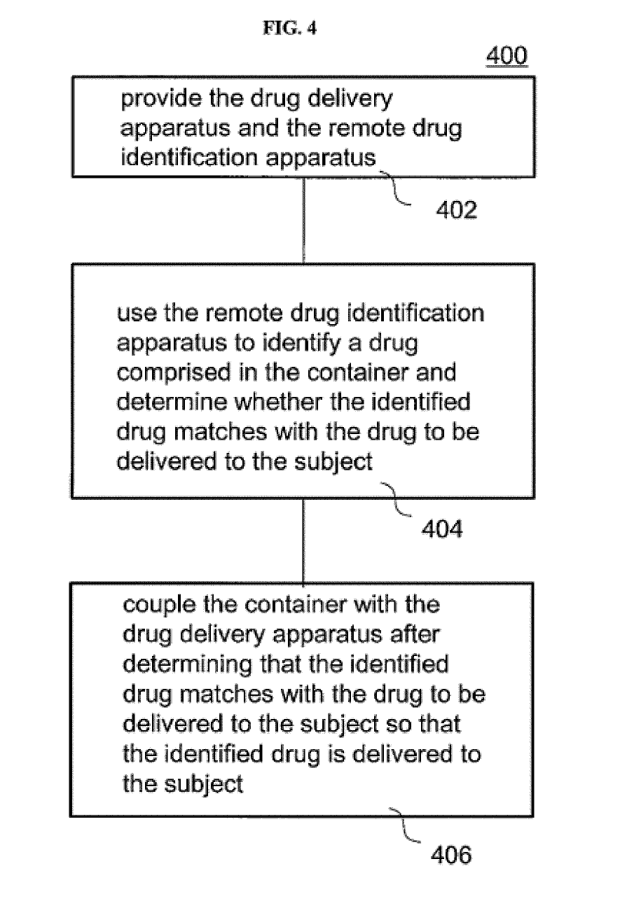

FIG. 4 is a schematic 400 showing a method of operating a drug delivery system according to various embodiments. The method may include, in 402, providing the drug delivery apparatus and the remote drug identification apparatus. The method may also include, in 404, using the remote drug identification apparatus to identify a drug comprised in the container and determine whether the identified drug matches with the drug to be delivered to the subject. The method may further include, in 406, coupling the container with the drug delivery apparatus after determining that the identified drug matches with the drug to be delivered to the subject so that the identified drug is delivered to the subject.

In other words, the remote drug identification apparatus may be used to determine the identity of the drug in the container. If the drug is identified to be the drug intended to be administered to the subject, the container is coupled to the drug delivery apparatus.

Using the remote drug identification apparatus to identify the drug comprised in the container may include scanning a label, such as a barcode label, on the container using a scanner of the remote drug identification apparatus to determine based on the label the identity of the drug comprised in the container.

The remote drug identification apparatus may further include a computing device configured to determine whether the identified drug comprised in the container matches with the drug that is to be delivered to the subject. In various embodiments, determining whether the identified drug matches with the drug to be delivered to the subject may include making a determination on whether the identified drug is a suitable drug for the subject, e.g. based on a history of drug allergies of the subject. The remote drug identification apparatus may retrieve the history of drug allergies of the subject from a database.

The remote drug identification apparatus may be configured to transmit the positive indication to the drug delivery apparatus upon the identified drug comprised in the container matches with the drug that is to be delivered to the subject. The flow controller of the drug delivery apparatus may switch or allow to be switched to an open state upon the communication module of the drug delivery apparatus receiving a positive indication from the drug identification apparatus.

The remote drug identification apparatus may be configured to provide an output, such as an audio output, a visual output or an audio-visual output, to provide details of the identified drug. The user, such as an anesthesiologist or a doctor administering the drug, may thus receive information such as whether the drug in the container matches with the drug to be administered to the subject, the dosage or concentration of the identified drug etc. The drug delivery apparatus provides a further check to ensure that the drug administered to the subject is indeed the correct one.

The method may also include decoupling the container and the drug delivery apparatus after delivering the drug to the subject. The coupling to and decoupling from the container may be carried out manually by the user.

The method may further include providing inputs on the drug delivered to the subject on the remote drug identification apparatus after decoupling the container and the drug delivery apparatus. The inputs may be manually inputted directly or indirectly (via another computing device or terminal) into the remote drug identification apparatus. For instance, the user may input or confirm that the drug has been delivered or administered, and/or the drug name, and/or the dosage administered to the subject. The inputs may be transmitted to the database such as Anesthesia Electronic Records database. Various embodiments may therefore provide an effective means of providing or updating a record of drug administration. The record may be useful as a future reference for medical professionals to know the drugs, dosage etc. that has been administered to the subject.

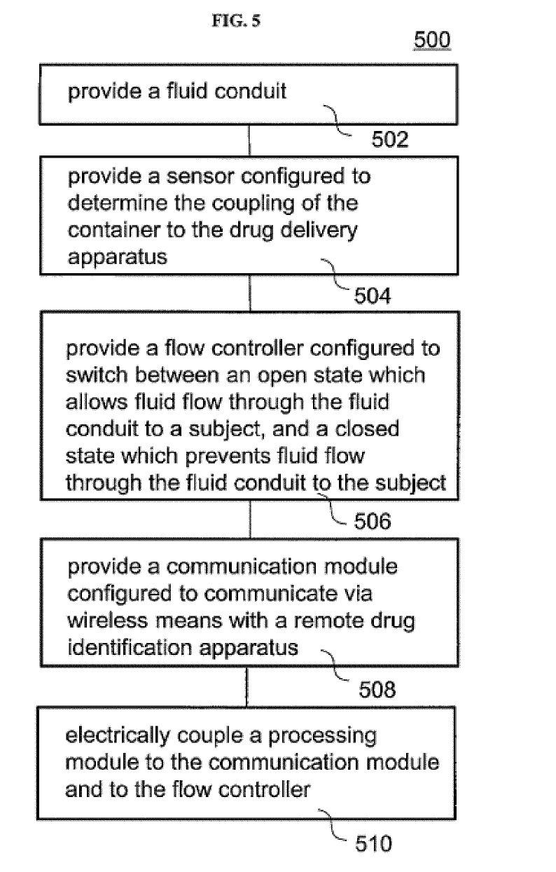

FIG. 5 is a schematic 500 illustrating a method of forming a drug delivery apparatus according to various embodiments. The method may include, in 502, providing a fluid conduit. The method may also include, in 504, providing a sensor configured to determine the coupling of the container to the drug delivery apparatus. The method may further include, in 506, providing a flow controller configured to switch between an open state which allows fluid flow through the fluid conduit to a subject, and a closed state which prevents fluid flow through the fluid conduit to the subject. The method may additionally include, in 508, providing a communication module configured to communicate via wireless means with a remote drug identification apparatus. The method may further include, in 510, electrically coupling a processing module to the communication module and to the flow controller. The flow controller may be configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus. The flow controller may be configured to be switched to the closed state after receiving a trigger from the sensor upon the container being decoupled from the drug delivery apparatus.

In other words, the method of forming the drug delivery apparatus may include providing a fluid conduit, a sensor, a flow controller, a communication module and a processing module. The processing module may be coupled to the communication module and the flow controller.

The sensor may be electrically coupled to the processing module and/or to the flow controller.

In various embodiments, a method of forming a drug delivery system may be provided. The method may further include forming or providing a drug delivery apparatus, and forming or providing the remote drug identification apparatus. The drug delivery system may be coupled to the drug identification apparatus via wireless means.

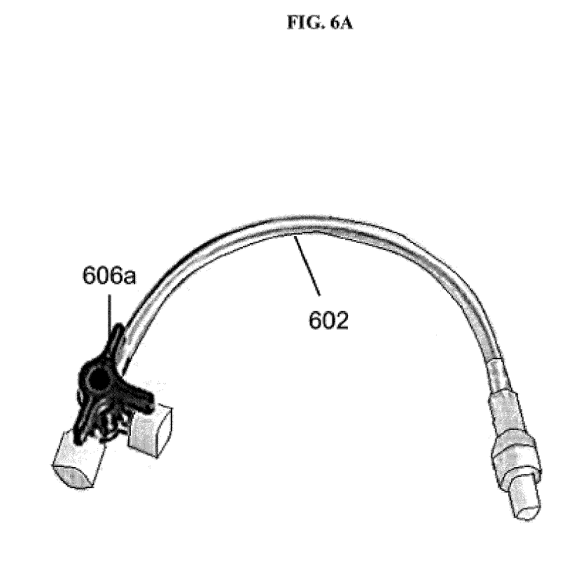

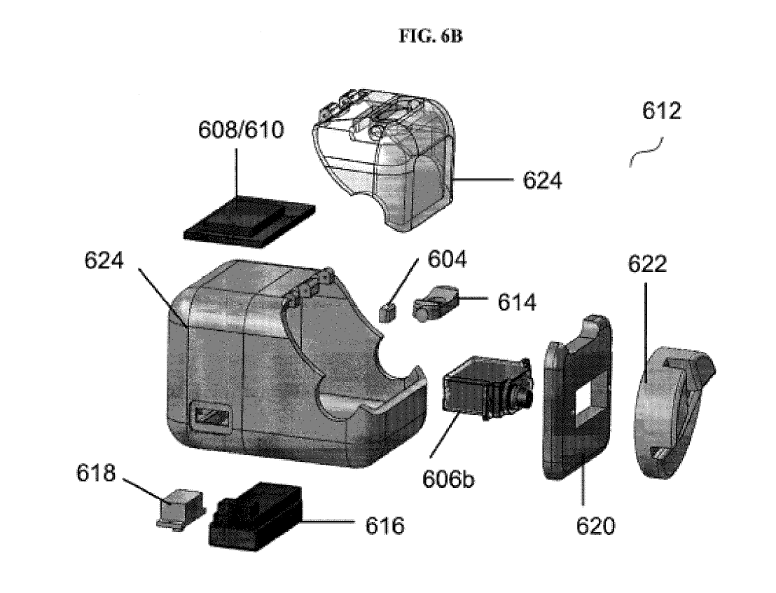

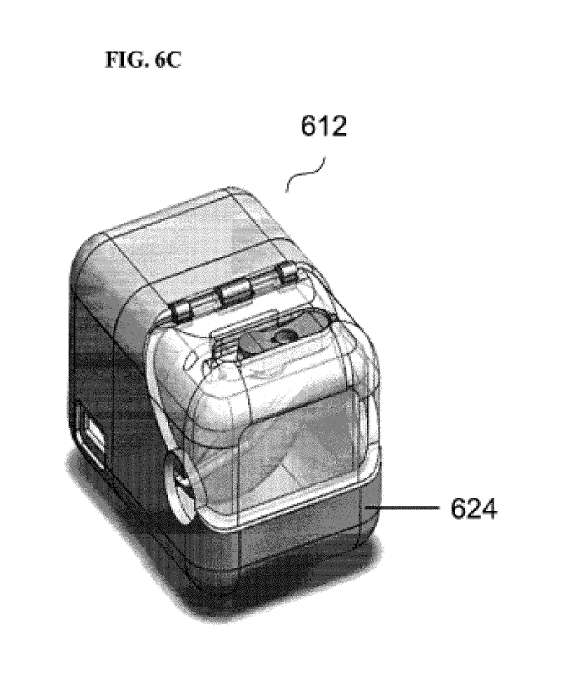

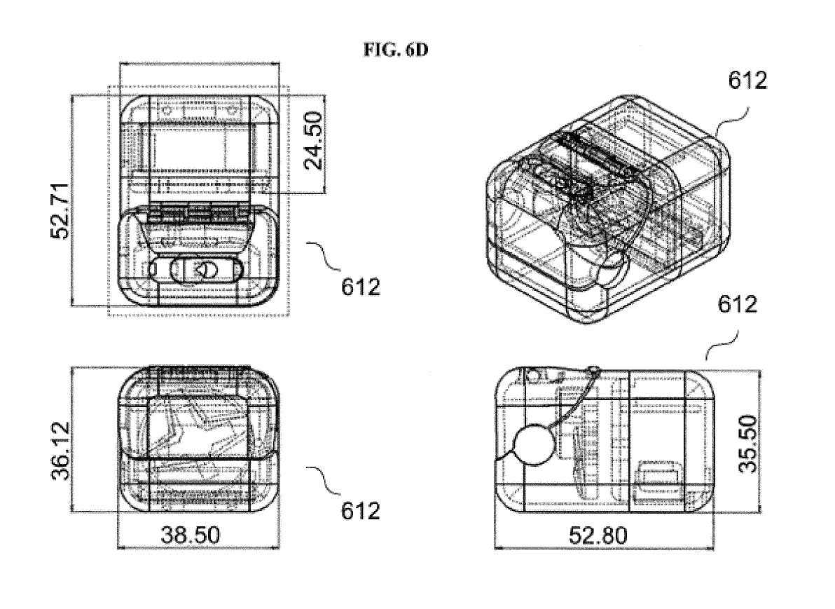

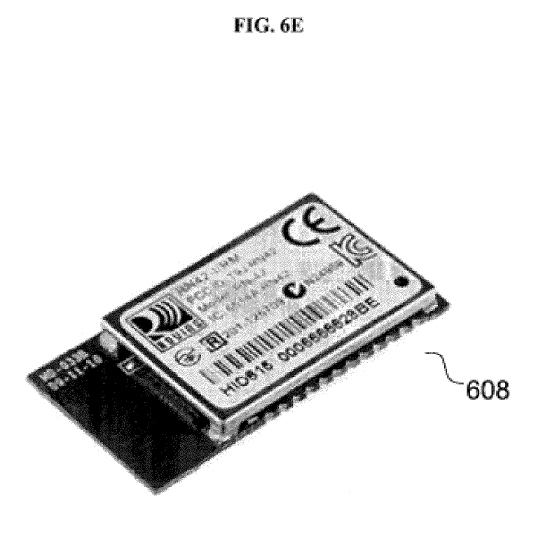

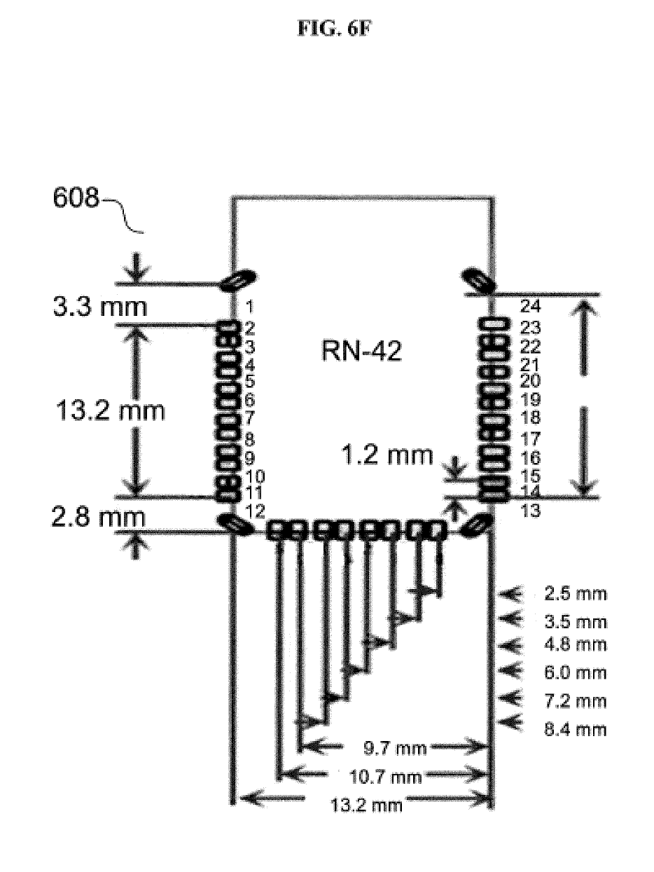

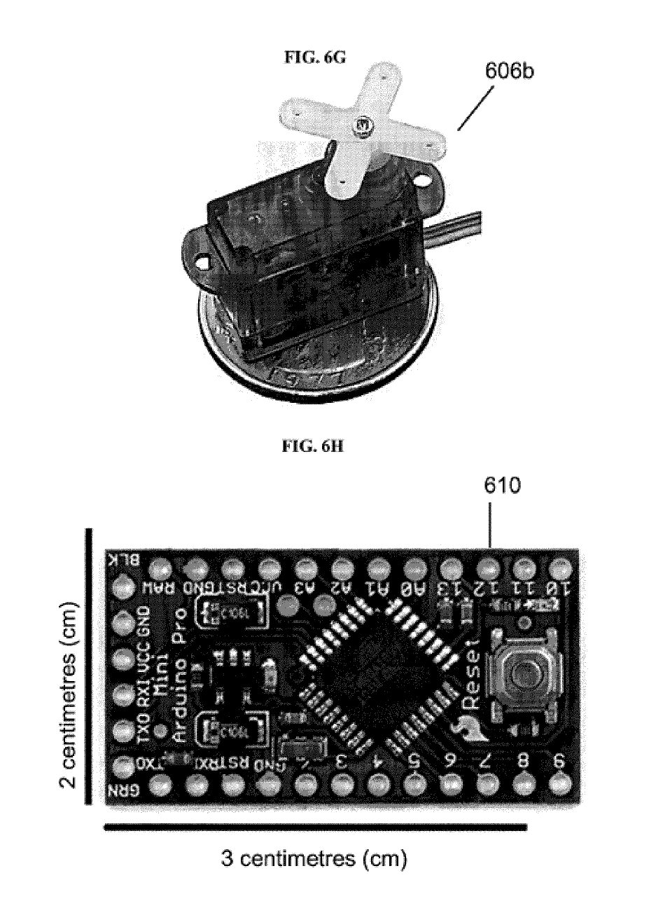

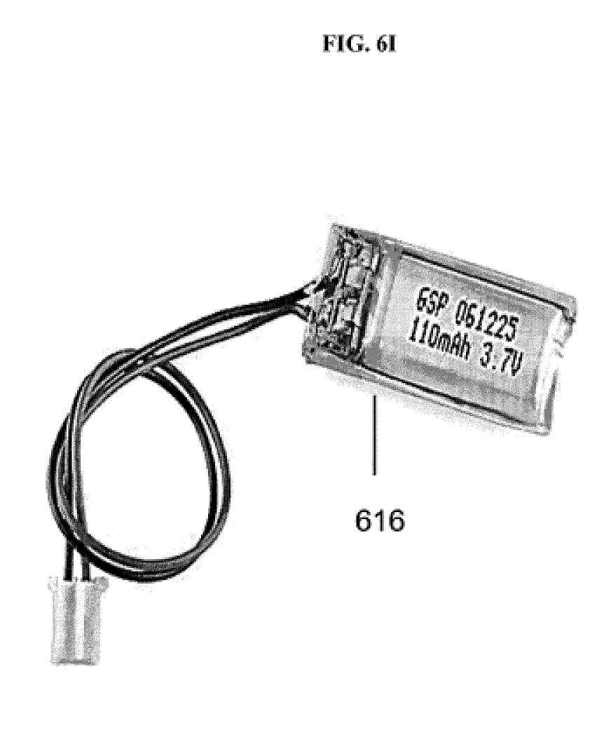

FIG. 6A shows a fluid conduit 602 and a flow control valve 606a of a drug delivery apparatus according to various embodiments. The fluid conduit 602 may be a disposable plastic intravenous tubing. The flow control valve 606a may be attached to the disposable plastic intravenous tubing 602.

FIG. 6B shows various components included in a housing unit 612 of the drug delivery apparatus according to various embodiments. The housing unit 612 may be configured to operate with the fluid conduit 602 and the flow control valve 606a shown in FIG. 6A. The housing unit 612 may include an infrared optical interrupter sensor 604 and a slider 612. The housing unit 612 may also include a communications module 608 such as a Bluetooth module, and a processing module 610 such as an Arduino microcontroller. The Bluetooth module and the Arduino microcontroller may be integrated as a single unit with an indicator light emitting diode. The integrated unit may be configured to receive Bluetooth signals from an external electronic device such as a personal computer (PC) running a customized software. The PC may be coupled to a barcode scanner. The integrated unit may also be configured to transmit Bluetooth signals to the external electronic device after drug delivery and the container is decoupled from the drug delivery apparatus.

The housing unit 612 may further include an actuator such as a servo motor 606b. The servo motor 606b may be configured to actuate the flow control valve 606a, i.e. to open the flow control valve 606a and to close the flow control valve 606a. The servo motor 606b and the flow control valve 606a may form the fluid controller. The flow controller may be in a closed state when the control valve 606a is closed, while the flow controller may be in an open state when the flow control valve 606a is opened. When the communications module 608 receives a positive indication from the drug identification apparatus, the communications module 608 may inform the processing module 610. The processing module 608 may then transmit a control signal to the servo motor 606b to actuate the flow control valve 606a to open for administration of the drug. After drug administration and the container, e.g. a syringe, is decoupled from the drug delivery apparatus, the processing module 608 may transmit a further control signal to the servo motor 606b to actuate the flow control valve 606a to close.

The housing unit 612 may further include a sensor 602 such as an infrared optical interrupter sensor. The infrared optical interrupter sensor 602 may fit onto a drug administration port of the plastic intravenous tubing 602. When the container such as a syringe containing the drug is coupled with the drug delivery apparatus, the infrared optical interrupter sensor 602 may be activated, and when the container is decoupled from the drug delivery apparatus, the infrared optical interrupter sensor 602 may be deactivated. The infrared optical interrupter sensor 602 may thus sense the coupling and decoupling between the container and the drug delivery apparatus. The housing unit 612 may also include a slider unit 614 in which the user may slide to expose the drug administration port for coupling of the container.

The housing unit 612 may also include a battery and charging unit 616 for providing power supply to the other components such as the integrated unit including the Bluetooth module and the Arduino microcontroller, as well as the infrared optical interrupter sensor 602. The battery and charging unit 616 may be electrically coupled to the integrated unit. In addition, the housing unit 612 may include a switch 618 for switching on and switching off the drug delivery apparatus. The housing unit 612 also includes the servo holder 620 for holding or securing the servo motor 606b. In addition, the housing unit 612 may further include a stopcock holder 622 for coupling to the plastic intravenous tubing 602 and a fluid bag.

The housing unit 612 may also include a casing 624 to house the various components. FIG. 6C shows the assembled housing unit 612 of the drug delivery apparatus according to various embodiments. FIG. 6D shows the technical drawings of the housing unit 612 according to various embodiments. The dimensions of the housing unit 612 are denoted in centimeters (cm). The housing unit 612 may be a re-usable housing.

FIG. 6E shows a photo of a Bluetooth module 608 included in the drug delivery apparatus according to various embodiments. FIG. 6F shows a schematic of the Bluetooth module included in the drug delivery apparatus according to various embodiments.

FIG. 6G shows images of the side view (left) and the perspective view (right) of the servo motor 606b of the drug delivery apparatus according to various embodiments.

FIG. 6H shows the Arduino microcontroller 610 included in the drug delivery apparatus according to various embodiments.

FIG. 6I shows the battery and charging unit 616 included in the drug delivery apparatus according to various embodiments.

During use, a fluid bag may be fluidically coupled or connected to an inlet of the disposable plastic intravenous tubing 602, e.g. through a stopcock holder. An outlet of the tubing may be attached to an intravenous line of the subject. When the flow controller is in a closed state, the flow control valve 606a may prevent the flow of the drug from a container, e.g. a syringe, into a drug administration port on the tubing, although fluid from the fluid bag may flow from the inlet through the tubing 602 to the outlet. When the flow controller is in an open state, the flow control valve 606a may allow the flow of the drug from the container into the drug administration port and to the outlet of the tubing 602.

FIG. 6J shows a drug identification apparatus 626 configured to work with the drug delivery apparatus according to various embodiments. The drug identification apparatus 626 may include a personal computer 628 and a barcode scanner 630 coupled to the personal computer 628. The personal computer 628 may include a customized software, which may be written using a suitable programming language such as C#. The barcode scanner 630 may be an over-the-shelf scanner, such as an Omni Directional 2D barcode scanner. When the barcode on a drug container or syringe is scanned, the customized software may determine, based on the barcode, the identity of the drug in the container or syringe, and to determine whether the drug in the container or syringe is the correct drug to be administered to the subject. The customized software may interpret and analyze the barcode. If the software determines that the drug is indeed the correct drug to be administered to the subject, the software may control the personal computer to send a Bluetooth signal providing a positive indication that the drug in the container is the correct drug to the drug delivery apparatus.

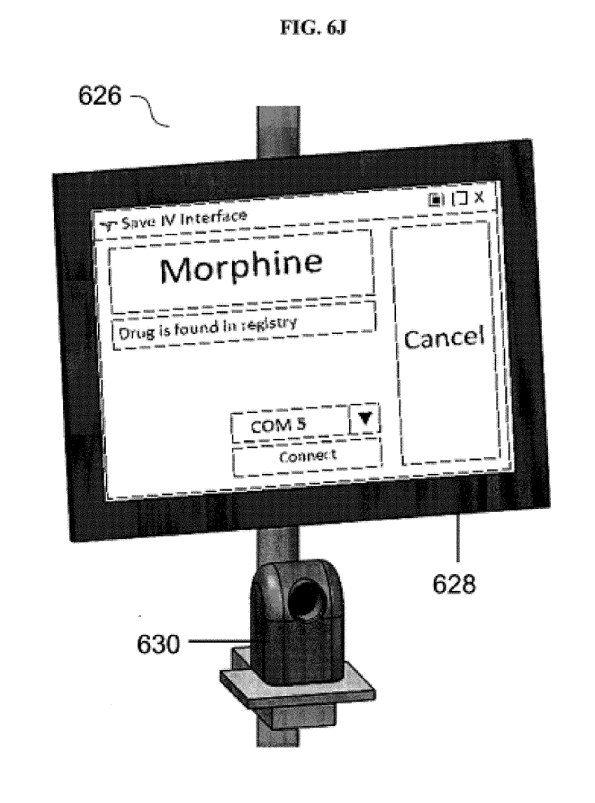

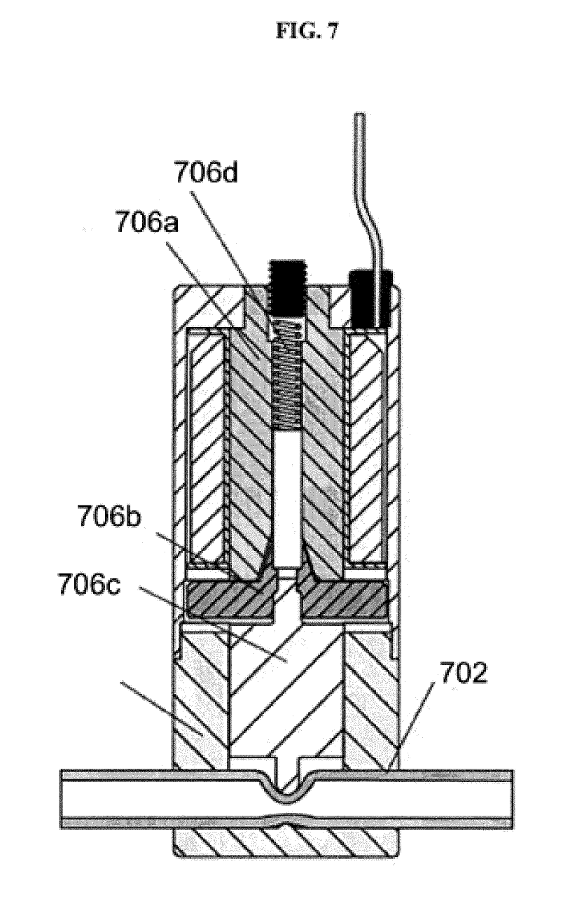

FIG. 7 shows a pinch valve of a drug delivery apparatus according to various embodiments. The drug delivery apparatus may include a solenoid 706a, an armature 706b, and a plunger 706c. A Bluetooth receiver-transmitter module (not shown in FIG. 7) may trigger a processing module (not shown in FIG. 7) to transmit a control signal to the solenoid 706a upon the Bluetooth receiver-transmitter module receiving a positive indication from a drug identification apparatus. When the processing module transmits the control signal, an electrical current may flow through the solenoid 706a to generate a magnetic field. The magnetic field may cause the armature 706b to move up from a first position to a second position. When the armature 706b moves from the first position to the second position, the plunger 706c attached or coupled to the armature 706b, may move, thus removing or reducing a ‘pinch’ or constriction along the tubing 702, thereby allowing fluid flow along the tubing 702. The movement of the armature 706 from the first position to the second position may also compress the spring 706d. When a sensor, e.g. a syringe-drug administration port connection sensor (not shown in FIG. 7) detects that the container is decoupled from the drug delivery apparatus, the sensor may trigger the stop of the flow of the electrical current through the solenoid 706a. The sensor may trigger the processing module, which may in turn transmit a further control signal to stop the flow of the electrical current through the solenoid 706a. The cessation of flow of the electrical current through the solenoid 706a may cause solenoid 706a to cease the generation of the magnetic field. As there is no longer a force due to the magnetic field acting on the armature 706b to compress the biasing spring 706c, the biasing spring 706c may return to its original length and may move the armature 706b down, thereby causing or increasing the ‘pinch’ or constriction along the tubing 702 and preventing fluid flow along the tubing 702.

When fluid flow along the tubing 702 is prevented, the pinch valve may be in the closed state, and when fluid flow along the tubing 702 is allowed, the pinch valve may be in the open state.

In various embodiments, the drug delivery apparatus may include an electromagnet configured to mechanically close a fluid conduit, e.g. a tubing, so that fluid flow through the fluid conduit is prevented, or mechanically open the fluid conduit so that fluid flow through the fluid conduit is allowed. A Bluetooth receiver-transmitter module may trigger a processing module to transmit a control signal to the electromagnet upon the Bluetooth receiver-transmitter module receiving a positive indication from a drug identification apparatus. When the processing module transmits the control signal, the electromagnet may mechanically open the fluid conduit to allow fluid flow through the fluid conduit. When a sensor, e.g. a syringe-drug administration port connection sensor detects that the container is decoupled from the drug delivery apparatus, the sensor may trigger the electromagnet to mechanically close the fluid conduit to prevent fluid flow through the fluid conduit. The sensor may transmit the trigger to the processing module, which may in turn control the electromagnet, e.g. via a further control signal, to mechanically close the fluid conduit.

When fluid flow along the fluid conduit is prevented, the flow controller including the electromagnet may be in the closed state, and when fluid flow along the fluid conduit is allowed, the flow controller including the electromagnet may be in the open state.

In various embodiments, the sensor may be a mechanical sensor. The mechanical sensor may be activated by the coupling or attachment of the container (e.g. syringe) with the drug delivery apparatus (e.g. the drug administration port), and may be deactivated by the decoupling of the container from the drug delivery apparatus (e.g. the drug administration port).

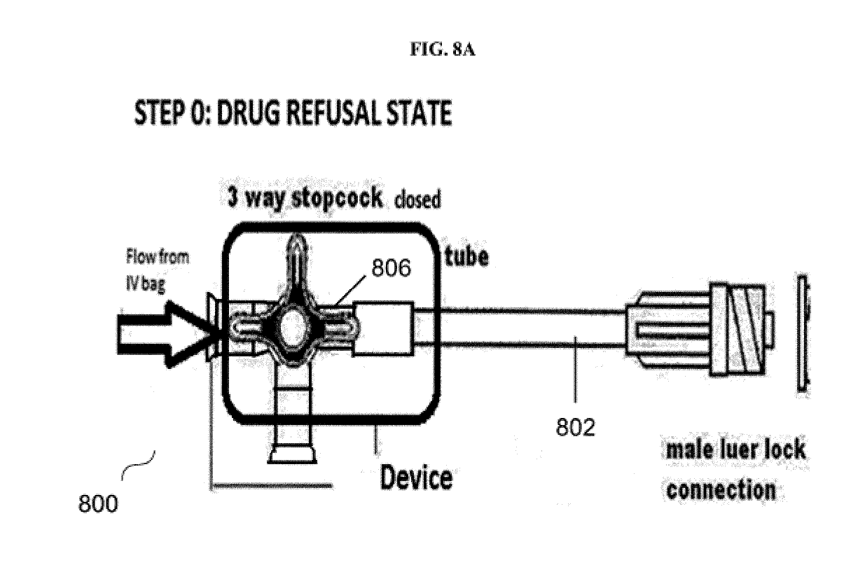

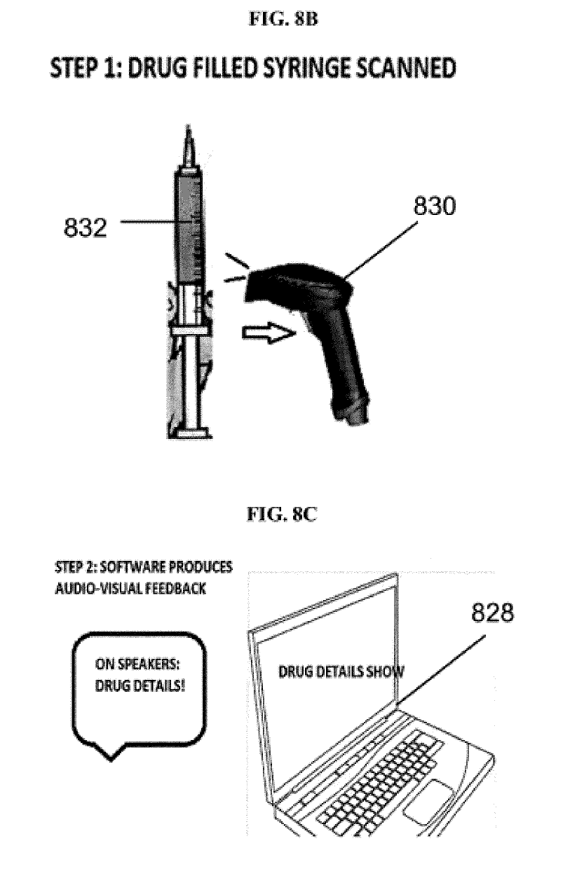

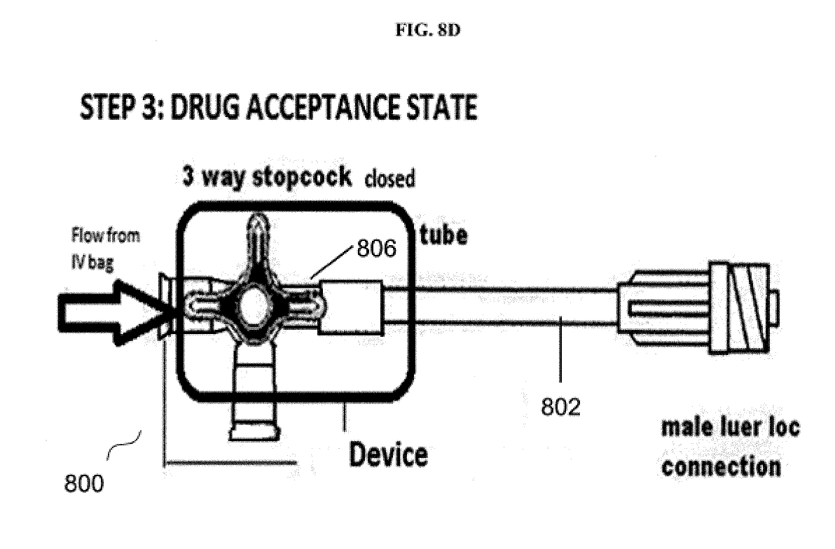

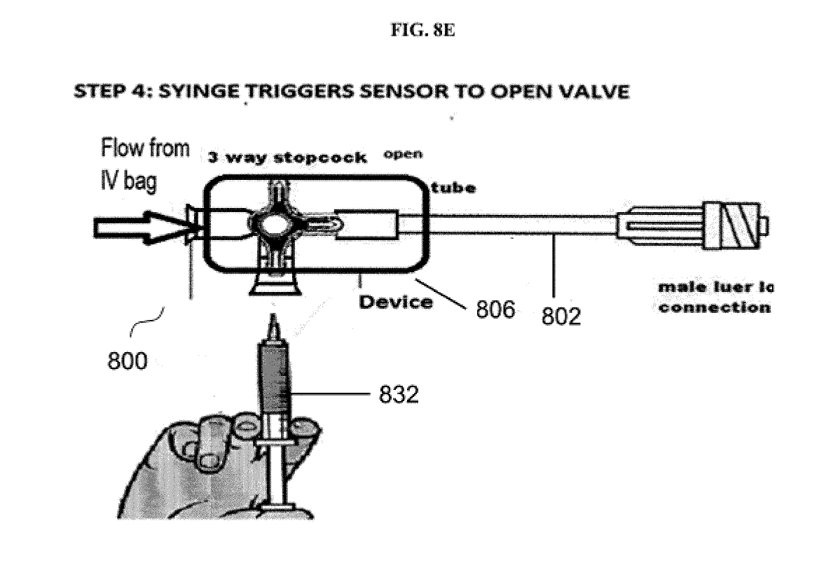

FIG. 8A shows the drug delivery apparatus 800 according to various embodiments with the flow controller 806 in a closed state. When the flow controller 806 is in the closed state, no fluid may flow through the tubing 802. The drug delivery apparatus 800 may be in a drug refusal state.

FIG. 8B shows a barcode scanner 830 being used to scan a barcode on a syringe 832 according to various embodiments. FIG. 8C shows a computing device 828 with a software being used to identify the drug based on the barcode and to generate an audio-visual indication containing the name of the drug as well as the concentration of the drug according to various embodiments. The computing device 828 may be coupled to the barcode scanner 830 shown in FIG. 8B.

FIG. 8D shows the drug delivery apparatus 800 according to various embodiments in a drug acceptance state after the computing device 828 with the software transmitting a signal containing a positive indication to the drug delivery apparatus 800. The drug delivery apparatus 800 may be configured to be in a drug acceptance state for a predetermined period of time, e.g. 10 seconds. If a sensor, e.g. an infrared photo-interrupter sensor of the drug delivery apparatus 800 is not triggered within the predetermined period of time (by coupling the syringe 832 to the drug delivery apparatus 800), the drug delivery apparatus 800 may switch back the drug refusal state. The sensor may be arranged at a drug administration port of the drug delivery apparatus 800. The flow controller 806 may remain in a closed state.

FIG. 8E shows the syringe 832 being brought into proximity of the drug delivery apparatus 800 according to various embodiments. When the syringe 832 is brought within a predetermined distance e.g. within 5 cm, of the drug delivery apparatus 800, the sensor may sense the syringe 832 and may trigger a motor of the drug delivery apparatus 800 to open the flow controller 806, e.g. a three-way valve, i.e. switch the flow controller 806 to an open state, if the drug delivery apparatus 800 is in the drug acceptance state. The flow controller 806 may remain in the open state as long as the sensor senses that the syringe is within the predetermined distance of the drug delivery apparatus 800.

When the syringe 832 is moved away from the drug delivery apparatus 800 outside of the predetermined distance, the sensor may cause the drug delivery apparatus 800 to revert to the drug refusal state. The flow controller 806 may switch back to the closed state.

The sensor may be activated when the syringe 832 is brought within a predetermined distance from the drug delivery apparatus 800, and may be deactivated when the syringe 832 is brought outside of the predetermined distance.

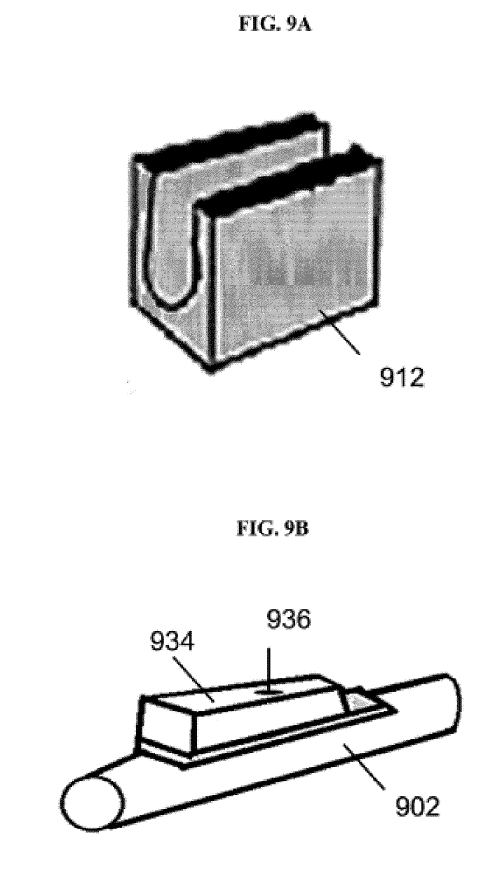

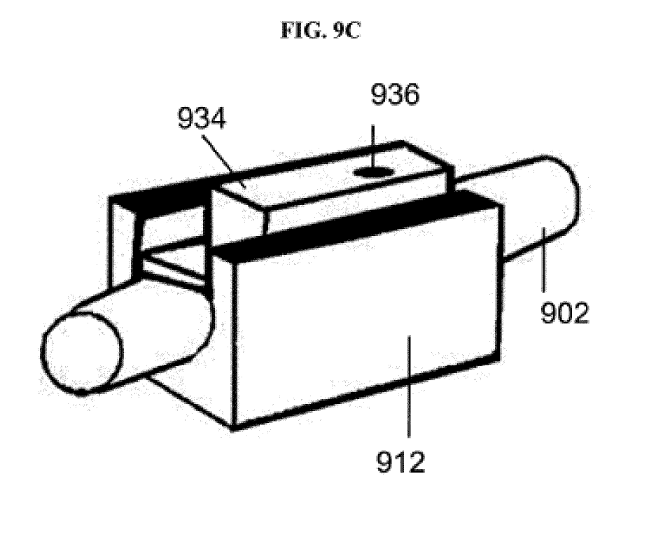

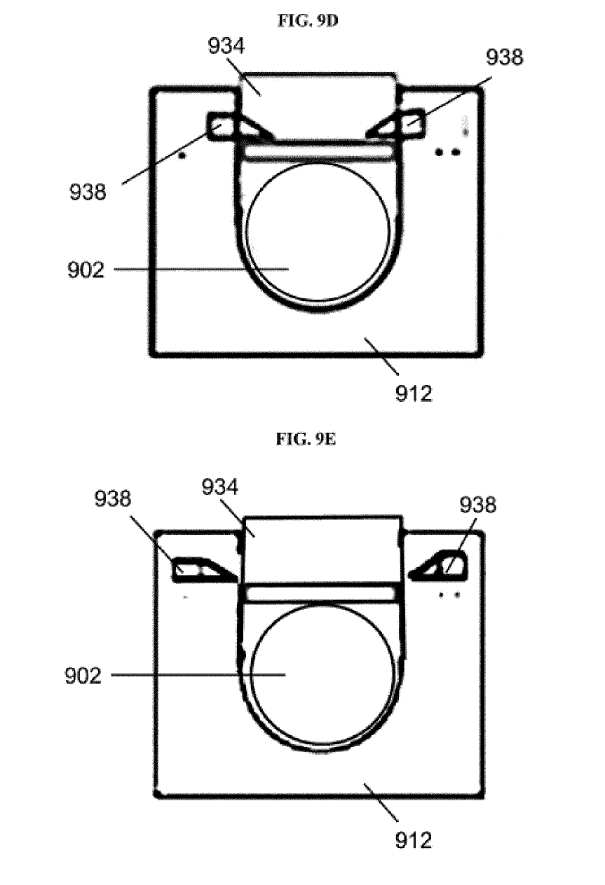

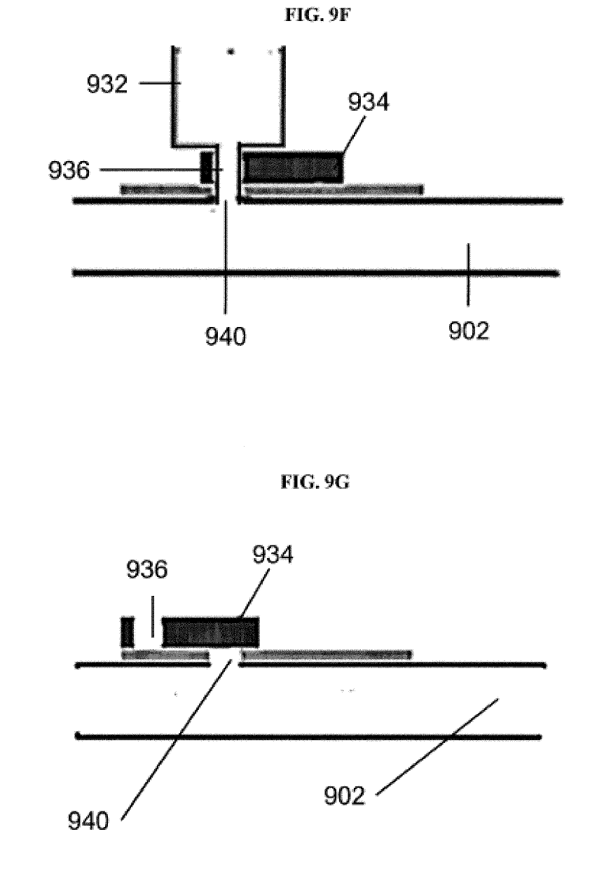

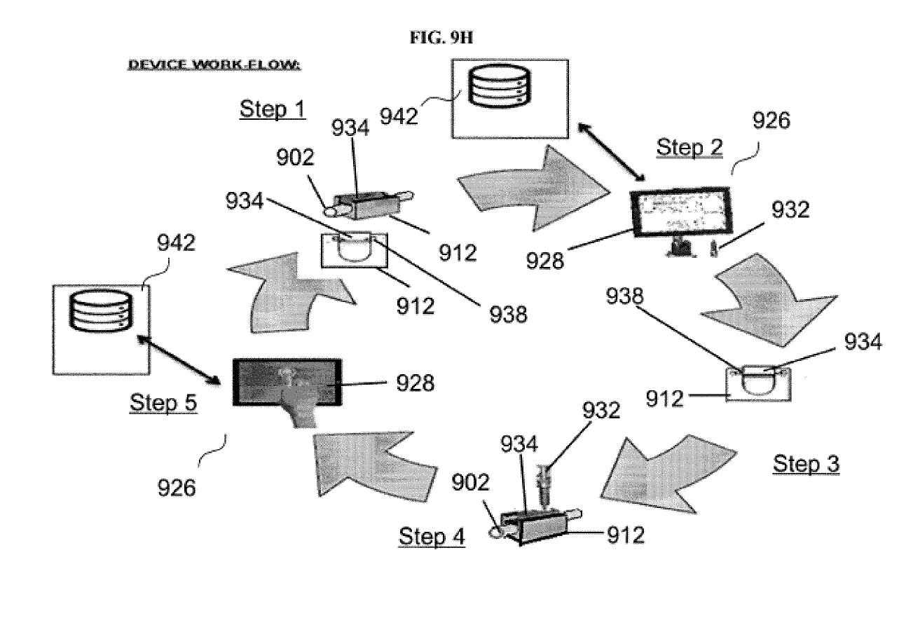

FIG. 9A is a housing unit 912 of a drug delivery apparatus according to various embodiments. The housing unit 912 may be a re-usable housing unit. The housing unit 912 may include a processing module such as a micro-controller, and a communication module such as a Bluetooth receiver-transmitter module. The Bluetooth receiver-transmitter module may be an over-the-shelf component. The housing unit 912 may further include a fluid controller. The flow controller may include a locking mechanism. The housing unit 912 may further include a battery to power the other components.

FIG. 9B shows a customized disposable tubing of the drug delivery apparatus according to various embodiments. The customized disposable tubing may include a tubing 902 which serves as a fluid conduit, and a movable slider cover 934. The movable slider cover 934 may include an opening 936 extending through the height of the slider cover 934, i.e. the opening 936 may be a through-hole. FIG. 9C shows the integration of the housing unit 912 and the customized disposable tubing according to various embodiments.

The housing unit 912 may also include a locking mechanism. The locking mechanism may include pins 938. FIG. 9D shows a cross-sectional schematic of the housing unit 912 and the customized disposable tubing in which the pins 938 are engaged with the slider cover 934 of the drug delivery apparatus according to various embodiments. FIG. 9E shows a cross-sectional schematic of the housing unit 912 and the customized disposable tubing in which the pins 938 are disengaged from the slider cover 934 of the drug delivery apparatus according to various embodiments. When the pins 938 are engaged with the slider cover 934, the slider cover 934 may be immovable. The slider cover 934 may be movable when the pins 938 are disengaged from the slider cover 934. In various embodiments, the locking mechanism may be a motorized locking mechanism. In various embodiments, the housing 912 may include a motor configured to control the pins 938. The motor may be electrically coupled to the processing module.

FIG. 9F is a schematic of a side view of the slider cover 934 and the customized disposable tubing in which the cover is at a first position so that the opening of the cover 934 is in fluidic communication with the port 940 on the tubing 902 according to various embodiments. FIG. 9G is a schematic of a side view of the slider cover 934 and the customized disposable tubing in which the cover is at a second position so that the opening of the cover is not in fluidic communication with the port 940 on the tubing 902 according to various embodiments. The port 940 may be a drug administration port. When the slider cover 934 is at the first position, the opening 936 of the slider cover 934 and the port 940 may be aligned, and fluid flow from the container 932 through the tubing 902 to the subject may be allowed. The fluid controller including the slider cover 934 may be in the open state. The drug from container 932, e.g. a syringe, may be delivered to the subject when the container 936 is coupled to the opening 936, thus achieving administration of the drug. When the container 932 is decoupled from the opening 936, a spring mechanism or spring (not shown in FIGS. 9F and 9G) may sense the decoupling of the container 932 from the opening 936 and move the slider cover 934 to a second position.

When the slider cover 934 is at the second position, the opening 936 of the slider cover 934 and the port 938 may be mis-aligned, and fluid flow into and through the tubing 902 to the subject may be prevented. The drug from container 936 may thus not be delivered to the subject. The fluid controller including the slider cover 934 may be in the closed state.

When the motor receives a control signal transmitted by the processing module, the motor may control the pins 938 to disengage from the slider cover 934. The slider cover 934 may then be moved manually from the second position to the first position for administration of the drug. After administration of the drug and the slider cover 934 is moved back to the second position by the spring, the processing module may transmit a further control signal to the motor. When the motor receives the further control signal transmitted by the processing module, the motor may control the pins 938 to engage the slider cover 934 to lock the slider cover 934.

The drug delivery apparatus with portions shown in FIGS. 9A-G may be used with the drug identification apparatus similar to that shown in FIG. 6I. A computer such as a tablet personal computer may read the contents of the label on the container or syringe 932 using an attached barcode scanner, counter-check the medication to be injected with drug allergies of the subject stored in a database such as the Anesthesia Electronics Records, and generate an audio-visual indication of the drug details. The visual indication may include an indication on the Graphics User Interface (GUI) of the software running on the computer. The computer with the software running may transmit a wireless signal containing a positive indication to the communication module of the drug delivery apparatus. The communication module may be electrically coupled to a microcontroller and may inform the microcontroller upon receiving the positive indication. The microcontroller may then send a control signal to an actuator to control the pins 938 to disengage from the slider cover 934. After the container 932 is decoupled from the drug delivery apparatus, the microcontroller may provide via the communication module an indication of completion of drug delivery to the drug identification apparatus, and the drug identification apparatus may transmit details such as the name of the drug administered, and dosage of the drug administered to the Anesthesia Electronics Records, upon manual confirmation by the user.

FIG. 9H shows a method of operating a drug delivery system including a drug delivery apparatus and a drug identification apparatus 926 according to various embodiments. The drug delivery apparatus may be the drug delivery apparatus as shown in FIG. 9H. In step 1, the slider cover 934 may be closed and the pins 938 may be engaged with the slider cover 934. In step 2, an user such as a doctor or an anesthesiologist may scan the barcode on the syringe 932 using the drug identification apparatus 926. A customized software running on a table personal computer 928 of the drug identification apparatus 926 may interpret the barcode and cross-check with a database 942 called the Anesthesia Electronics Records. The computer 928 may then announce the details of the drugs and send a signal to a micro-controller of the drug delivery apparatus via a communication nodule of the drug delivery apparatus. Upon receiving the signal, the microcontroller may then control a motor to actuate the pins 938 to disengage from the slider cover 934 as shown in step 3. In step 4, the user may move the slider cover 934 so that the opening of the slider cover 934 may align with a drug administration port of the tubing 902, and may couple or attach the syringe 932 to the port of the tubing 902. In step 5, after the completion of drug delivery, the drug delivery apparatus may provide an indication showing completion of the drug administration to the drug identification apparatus 926. The user may confirm the drug name and dose on the GUI shown on the computer 928. The confirm details may be transmitted to the Anesthesia Electronics Records 942 through a data bridge. The slider cover 934 may be moved so that the opening is mis-aligned with the port, and the pins 938 may be controlled by the motor to engage the slider cover 934 again as shown in Step 1.

In various embodiments, the drug delivery apparatus may include a flow sensor configured to determine or assess the dose of the drug administered. The incorporation of a flow sensor may also address medication errors due to incorrect dosage (which occurs in about 25% to about 35% of medication errors) and may further reduce frequency of anesthesia medication errors. The software for recording drug administration may also be modified for use in drug inventory management and for accurate hospital billing purposes, which may reduce healthcare costs and further add value.

Various embodiments may address a major cause of anesthesia medication errors. Various embodiments may see widespread adoption, may become a standard part of OT anesthesia safety equipment and/or may play a significant role in improving patient safety.

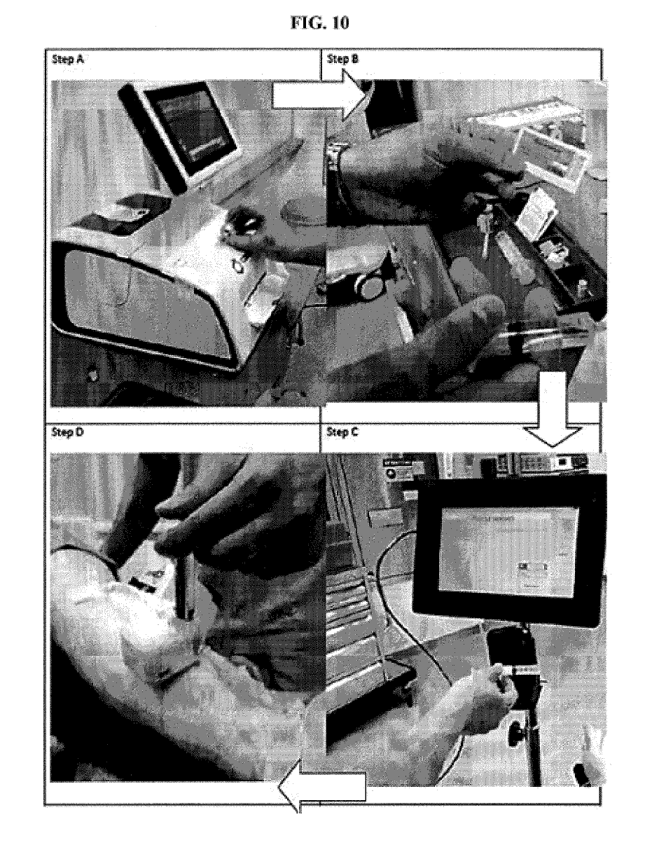

FIG. 10 shows images of a prototype system according to various embodiments used in conjunction with a Codonics SLS Label printer. In Step A, the drug ampoule is scanned, and a syringe label is printed using the Codonics SLS Label printer. In Step B, the the printed syringe label is applied on a syringe. In Step C, the printed label on the syringe is scanned with a prototype scanner, which provides an audio-visual cue consisting of the name of the drug and concentration of the drug. In Step D, the prototype drug delivery apparatus may be unlocked and the syringe may be coupled with the drug administration port of the prototype drug delivery apparatus.

The device prototype system was tested in the Singhealth Academia Simulation operating theatre (OT). The simulated OT environment resembles a real operating theatre and includes a manikin with an intravenous fluid drip set attached to a drug administration port for delivery to the manikin's arm.

There were two groups involved in the testing: a Control group without the prototype device and an intervention group with the prototype device.

Each anesthesia intravenous drug administration event may involve a two-step safe drug administration procedure consisting of scanning the ampoule to generate a correctly labelled syringe, and scanning the drug-filled syringe prior to administration at the intravenous drug administration port. An intravenous drug administration event may be considered compliant if the two-step safe drug administration procedure is followed. The primary outcome may include compliance to the two-step safe drug administration procedure.

Secondary outcomes may include end-user acceptance, feedback from participant anesthesiologists, assessment of device fail-safe feature, and/or time taken to administer the intravenous drug.

The participants included two anesthesiologists from the Singapore General Hospital invited to participate in the study. These anesthesiologists were not informed about the study outcome being measured. Each anesthesiologist was asked to administer anesthesia for 10 simulated surgical cases (5 in Control group and 5 in intervention group) in the simulated OT. Compliance with the primary outcome was monitored by an independent observer.

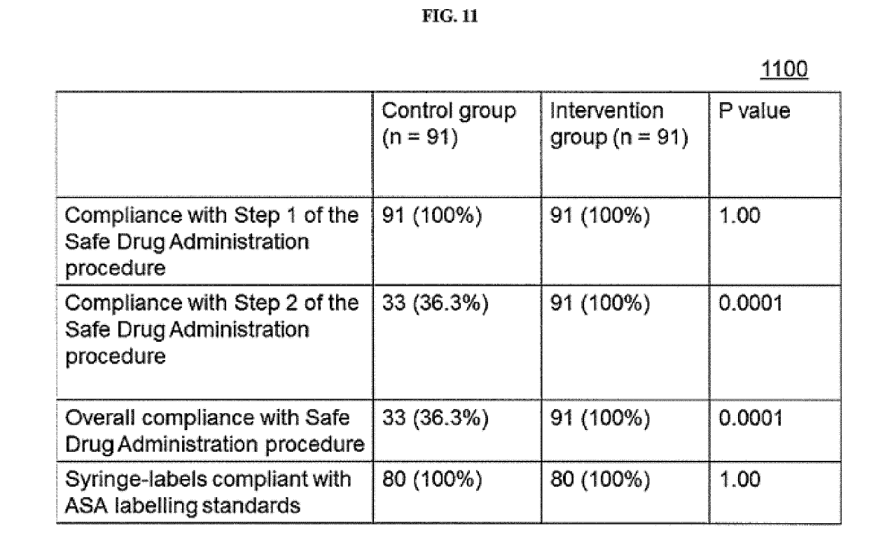

Previous studies suggest that primary outcome occurs only in up to 60% of intravenous drug administration events. To detect a difference of 20% in the primary outcome with power of 80% and an alpha error of 0.05, 91 intravenous drug administration events was carried out in each study group. Simulation cases were constructed such than each surgical case had about 9 intravenous drug administration events, so that to a sample size of 20 simulated surgical cases (10 cases in each group) were required. FIG. 11 is a table 1100 showing the results of the study.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A drug delivery apparatus for coupling with a container, the drug delivery apparatus comprising:
a fluid conduit;
a sensor configured to determine the coupling of the container to the drug delivery apparatus;
a flow controller configured to switch between an open state which allows fluid flow through the fluid conduit to a subject, and a closed state which prevents fluid flow through the fluid conduit to the subject;
a communication module configured to communicate wirelessly with a remote drug identification apparatus; and
a processing module electrically coupled to the communication module and to the flow controller;
wherein the flow controller is configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus; and
wherein the flow controller is configured to be switched to the closed state after receiving a trigger from the sensor upon the container being decoupled from the drug delivery apparatus.

2. The drug delivery apparatus according to claim 1,
wherein the sensor is a mechanical sensor.

3.-4. (canceled)

5. The drug delivery apparatus according to claim 1,
wherein the flow controller comprises a cover having an opening;
wherein the cover is at a first position in which the opening of the cover is in fluidic communication with a port on the fluid conduit when the flow controller is in the open state; and
wherein the cover is at a second position in which the opening of the cover is not in fluidic communication with the port on the fluid conduit when the flow controller is in the closed state.

6. The drug delivery apparatus according to claim 5,
wherein the flow controller further comprises a lock; and
wherein the processing module is configured to control the lock to disengage from the cover upon the communication module receiving the positive indication from the drug identification apparatus so that the cover is configured to be switched manually to the open state.

7. The drug delivery apparatus according to claim 1,
wherein the flow controller comprises a pinch valve configured to switch between the open state and the closed state; and
wherein the pinch valve is configured to be switched to the open state after receiving the control signal from the processing module upon the communication module receiving the positive indication from the drug identification apparatus.

8. (canceled)

9. The drug delivery apparatus according to claim 1,
wherein the flow controller is configured to remain in the closed state by the processing module upon the communication module receiving a negative indication from the drug identification apparatus that the container comprises another drug that is not intended to be delivered to the subject.

10. The drug delivery apparatus according to claim 1,
wherein the communication module is configured to transmit a signal to the remote drug identification apparatus indicating completion of drug delivery after the container is decoupled from the drug delivery apparatus.

11.-12. (canceled)

13. A drug delivery system comprising:
a remote drug identification apparatus; and
a drug delivery apparatus for coupling with a container, the drug delivery apparatus comprising:
a fluid conduit;
a sensor configured to determine the coupling of the container to the drug delivery apparatus;
a flow controller configured to switch between an open state which allows fluid flow through the fluid conduit to a subject, and a closed state which prevents fluid flow through the fluid conduit to the subject;
a communication module configured to communicate wirelessly with the remote drug identification apparatus; and
a processing module electrically coupled to the communication module and to the flow controller;
wherein the flow controller is configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus; and
wherein the flow controller is configured to be switched to the closed state after receiving a trigger from the sensor upon the container being decoupled from the drug delivery apparatus.

14. The drug delivery system according to claim 13,
wherein the remote drug identification apparatus comprises a scanner configured to scan a label on the container to determine, based on the label, an identity of a drug comprised in the container.

15.-16. (canceled)

17. The drug delivery system according to claim 14,
wherein the remote drug identification apparatus is configured to provide an output providing details of the identified drug.

18. A method of operating a drug delivery apparatus, the method comprising:
coupling the drug delivery apparatus with a container; wherein the drug delivery apparatus comprises:
a fluid conduit;
a sensor configured to determine the coupling of the container to the drug delivery apparatus;
a flow controller configured to switch between an open state which allows fluid flow through the fluid conduit to a subject, and a closed state which prevents fluid flow through the fluid conduit to the subject;
a communication module configured to communicate wirelessly with a remote drug identification apparatus; and
a processing module electrically coupled to the communication module and to the flow controller;
wherein the flow controller is configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus; and
decoupling the container and the drug delivery apparatus, wherein the flow controller is configured to be switched to the closed state after receiving a trigger from the sensor upon the container being decoupled from the drug delivery apparatus.

19. The method according to claim 18, further comprising:
coupling the drug delivery apparatus to the subject before coupling the container with the drug delivery apparatus.

20. A method of operating a drug delivery system, the method comprising:
providing a remote drug identification apparatus and a drug delivery apparatus for coupling with a container, the drug delivery apparatus comprising:
a fluid conduit;
a sensor configured to determine the coupling of the container to the drug delivery apparatus;
a flow controller configured to switch between an open state which allows fluid flow through the fluid conduit to a subject, and a closed state which prevents fluid flow through the fluid conduit to the subject;
a communication module configured to communicate wirelessly with the remote drug identification apparatus; and
a processing module electrically coupled to the communication module and to the flow controller;
wherein the flow controller is configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus; and
wherein the flow controller is configured to be switched to the closed state after receiving a trigger from the sensor upon the container being decoupled from the drug delivery apparatus;
using the remote drug identification apparatus to identify a drug comprised in the container and determine whether the identified drug matches with the drug to be delivered to the subject; and
coupling the container with the drug delivery apparatus after determining that the identified drug matches with the drug to be delivered to the subject so that the identified drug is delivered to the subject.

21. The method according to claim 20,
wherein using the remote drug identification apparatus to identify the drug comprised in the container comprises scanning a label on the container using a scanner of the remote drug identification apparatus to determine based on the label the identity of the drug comprised in the container.

22. The method according to claim 21,
wherein the remote drug identification apparatus further comprises a computing device configured to determine whether the identified drug comprised in the container matches with the drug that is to be delivered to the subject.

23. The method according to claim 22,
wherein the remote drug identification apparatus is configured to transmit the positive indication to the drug delivery apparatus upon the identified drug comprised in the container matches with the drug that is to be delivered to the subject.

24. The method according to claim 22,
wherein the remote drug identification apparatus is configured to provide an output providing details of the identified drug.

25. (canceled)

26. The method according to claim 20, further comprising:
decoupling the container and the drug delivery apparatus after delivering the drug to the subject.

27. (canceled)

28. A method of forming a drug delivery apparatus, the method comprising:
providing a fluid conduit;
providing a sensor configured to determine the coupling of the container to the drug delivery apparatus;
providing a flow controller configured to switch between an open state which allows fluid flow through the fluid conduit to a subject, and a closed state which prevents fluid flow through the fluid conduit to the subject;
providing a communication module configured to communicate wirelessly with a remote drug identification apparatus; and
electrically coupling a processing module to the communication module and to the flow controller;
wherein the flow controller is configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus; and
wherein the flow controller is configured to be switched to the closed state after receiving a trigger from the sensor upon the container being decoupled from the drug delivery apparatus.

29. A method of forming a drug delivery system, the method comprising forming a drug delivery apparatus for coupling with a container the drug delivery apparatus comprising:
a fluid conduit;
a sensor configured to determine the coupling of the container to the drug delivery apparatus;
a flow controller configured to switch between an open state which allows fluid flow through the fluid conduit to a subject, and a closed state which prevents fluid flow through the fluid conduit to the subject;
a communication module configured to communicate wirelessly with a remote drug identification apparatus; and
a processing module electrically coupled to the communication module and to the flow controller;
wherein the flow controller is configured to be switched to the open state after receiving a control signal from the processing module upon the communication module receiving a positive indication from the drug identification apparatus that the container comprises a drug that is to be delivered to the subject, so that the drug is allowed to flow from the container through the fluid conduit to the subject when the container is coupled to the drug delivery apparatus; and
wherein the flow controller is configured to be switched to the closed state after receiving a trigger from the sensor upon the container being decoupled from the drug delivery apparatus; and
forming the remote drug identification apparatus.
